(19) United States
(12) Patent Application Publication
(10) Pub. No.: US 2013/0211231 A1
(43) Pub. Date: Aug. 15, 2013

(54) METHOD AND SYSTEM FOR VISUALIZATION OF TREATMENT VOLUMES

(71) Applicants: Manivannan Sundarapandian, Bangalore (IN); Vijay Daniel Manason, Bangalore (IN)

(72) Inventors: Manivannan Sundarapandian, Bangalore (IN); Vijay Daniel Manason, Bangalore (IN)

(21) Appl. No.: 13/396,567

(22) Filed: Feb. 14, 2012

(51) Int. Cl. A61B 6/02; A61B 6/03; A61B 5/055

(52) U.S. Cl. 600/411; 600/407; 600/410

(57) ABSTRACT

A method for visualization of treatment volumes includes accessing a plurality of images of a region of interest in a subject that are parallel to a plane. The method also includes delineating a cross section of a first treatment volume and a cross section of a second treatment volume in each of the plurality of images. The method includes reconstructing a three dimensional representation of the first and second treatment volumes from the plurality of images based on the delineated cross sections in the plurality of images. The method includes displaying, in a three dimensional image, a portion of the first treatment volume that is located on a first side of a plane and displaying, in the three dimensional image, at least a part of a portion of the second treatment volume that is located on a second side of the plane. The second side is opposite the first side.

### FIELD

The present embodiments relate to a method and a system for improving visualization of treatment volumes for treatment planning.

### BACKGROUND

Pathological anatomies such as tumors and lesions are characterized by abnormal growth of tissue resulting from the uncontrolled, progressive multiplication of cells, while serving no physiological function. These pathological anatomies may also be malignant in some cases, requiring proper treatment.

Pathological anatomies may be treated with an invasive procedure, such as surgery. The invasive procedure may be harmful and full of risks for the patient. A non-invasive method to treat a pathological anatomy (e.g., a tumor, a legion, a vascular malformation, or a nerve disorder) is external beam radiation therapy.

Initially, treatment planning was generally carried out through a manual manipulation of standard isodose charts onto patient body contours generated by direct tracing or lead wire representation. The treatment planning relied heavily on the judicious choice of beam weight and wedging by an experienced dosimetrist.

The simultaneous development of computed tomography (CT), with the advent of readily accessible computing power, led to the development of CT based computerized treatment planning. CT based computerized treatment planning provides the ability to view dose distributions directly superimposed upon a patient's axial anatomy. The entire treatment planning process involves many steps, beginning with beam data acquisition and entry into the computerized TPS, through patient data acquisition, to treatment plan generation and the final transfer of data to the treatment machine.

One of the techniques is use of image-guided three-dimensional (3-D) radiotherapy that aims at delivering the largest possible and the most homogeneous radiation dose to a tumor target, while keeping the dose to surrounding normal tissues to a minimum. Thus, a main step in radiation therapy planning is to determine and to delineate a target volume in three dimensions in order to produce a treatment volume that will encompass the tumor and normal tissues with as much precision as possible.

Volume definition is a prerequisite for meaningful 3D treatment planning and for accurate dose reporting. The International Commission on Radiation Units and Measurements (ICRU) reports 50 and 62 define and describe several target and critical structure volumes that aid in the treatment planning process and that provide a basis for comparison of treatment outcomes. These volumes include a gross tumor volume (GTV), a clinical target volume (CTV), and a planning target volume (PTV). The gross tumor volume (GTV) is the gross palpable or visible/demonstrable extent and location of malignant growth. The clinical target volume (CTV) is the GTV plus a margin to include local subclinical tumor spread. The CTV may also include regional nodes. The GTV and the CTV are based on anatomic, biological and clinical considerations and do not account for the technical factors of treatment. The planning target volume (PTV) is the CTV plus a margin to provide that the prescribed dose is actually absorbed in the CTV.

As a part of the treatment planning, the treatment volumes may be visualized for determining the dose distribution, correctness of contouring and reviewing the plan, for example. The volumes are visualized by use of planning software that includes a two dimensional visualization. A user (e.g., physicians, medical physicists, or medical dosimetrists) may see a sectional view of these volumes. However, since the visualization is on one linear plane, the relative volumetric aspects are not shown explicitly.

In a three dimensional visualization, the user may get a volumetric perception. However, since the GTV is embedded in the CTV, which is embedded in the PTV, volumes may mask inner volumes. The transparency control may show the inner volume to an extent. This may confuse the user, because the user may not be able to clearly differentiate the volumes in all cases.

### SUMMARY

It is desirable to provide a technique for visualization of treatment volumes, such that the treatment volumes are clearly differentiable from each other. Furthermore, it is desirable to develop a system to implement the technique for visualization of treatment volumes.

In a first aspect, a method for visualization of at least a first treatment volume and a second treatment volume, the second treatment volume encompassing the first treatment volume, includes accessing a plurality of images of a region of interest in a subject. The plurality of images are parallel to a plane. Different images of the plurality of images represent different slices of the subject along a normal vector of the plane. The method includes delineating a cross section of the first treatment volume and a cross section of the second treatment volume in each of the plurality of images, and reconstructing a three dimensional representation of the first treatment volume and the second treatment from the plurality of images based on the delineated cross sections in the plurality of images. The method also includes displaying, in a three dimensional image, a portion of the first treatment volume that is located on a first side of a plane and displaying, in the three dimensional image, at least a part of a portion of the second treatment volume that is located on a second side of the plane. The second side is opposite the first side.

In a second aspect, a system for visualization of at least a first treatment volume and a second treatment volume, the second treatment volume encompassing the first treatment volume, includes a processor coupled to an image acquisition system for acquiring a plurality of images of a region of interest in a subject. The processor is configured for accessing a plurality of images of the region of interest in the subject. The plurality of images are parallel to a same plane. Different images of the plurality of images represent different slices of the subject along a normal vector of the plane. The processor is configured for delineating a cross section of the first treatment volume and a cross section of the second treatment volume in each image of the plurality of images, and reconstructing a three dimensional representation of the treatment volumes from the plurality of images based on the delineated cross sections in the plurality of images. The system also includes a display unit for displaying, in a three dimensional image, a portion of the first treatment volume that is located on a first side of a plane and displaying, in the three dimensional image, at least a part of a portion of the second treatment volume that is located on a second side of the plane. The second side is opposite the first side.

In a third aspect, a non-transitory computer readable medium is provided. The non-transitory computer readable medium includes instructions that, when executed by a processor, causes the processor to perform a method for visualization of at least a first and a second treatment volume, the second treatment volume encompassing the first treatment volume. The method includes accessing a plurality of images of a region of interest in a subject. The plurality of images are parallel to a same plane. Different images of the plurality of images represent different slices of the subject along a normal vector of the plane. The instructions also include delineating a cross section of the first treatment volume and a cross section of the second treatment volume in each of the plurality of images, and reconstructing a three dimensional representation of the treatment volumes from the plurality of images based on the delineated cross sections in the plurality of images. The instructions also include displaying, in a three dimensional image, a portion of the first treatment volume that is located on a first side of a plane and displaying, in the three dimensional image, at least a part of a portion of the second treatment volume that is located on a second side of the plane. The second side is opposite the first side.

### BRIEF DESCRIPTION OF THE DRAWINGS

FIG. 1 is a schematic representation of example treatment volumes in a medical image;

FIG. 2 is a flowchart illustrating an exemplary method of visualization of treatment volumes in medical images;

FIG. 3 illustrates a three dimensional view of example treatment volumes;

FIG. 4 is a schematic diagram of one embodiment of a system for the visualization of treatment volumes; and

FIG. 5 is a schematic diagram of a display unit depicting simultaneous display of example treatment volumes based on intensity and dose distribution.

### DETAILED DESCRIPTION OF THE INVENTION

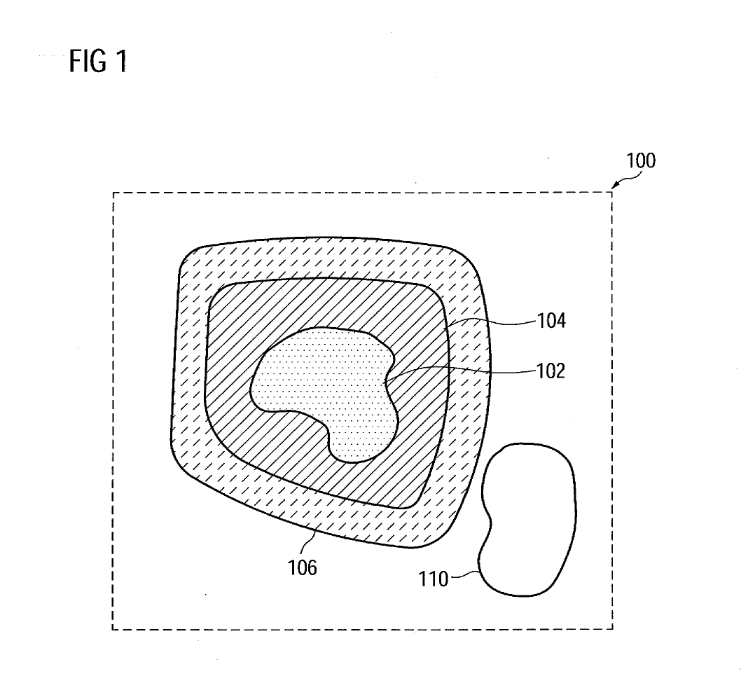

FIG. 1 is a schematic representation of an example medical image 100 depicting treatment volumes. The medical image 100 is a computed tomography (CT) image, a medical resonance (MR) image, a positron emission tomography image or a C-arm CT image, for example. The medical image 100 may be an image with a malformation. The medical image 100 may be acquired using an image acquisition system such as a CT, an MR, a PET, or a combination of any of the above mentioned imaging system. The medical image 100 may also be acquired using multi modality imaging systems that employ an imaging system in conjunction with other imaging modalities, position-tracking systems or other sensor systems.

As used herein, “malformation” may be an abnormal formation or development of a tissue or an organ. Malformation may include a tumor, a fibroid or any other malformation that may be benign or cancerous.

The medical image 100 shows different volumes (e.g., treatment volumes), such as a gross tumor volume 102, a clinical target volume 104, and a planning target volume 106, that are defined for treatment planning and for accurate dose reporting for treatment of the malformation. Reference numeral 110 represents an organ at risk that may be an organ susceptible to radiation dose. The gross tumor volume (GTV) 102 is a gross palpable or visible/demonstrable extent and location of malignant growth. The clinical target volume 104 (CTV) is the GTV 102 plus a margin to include local subclinical tumor spread. The GTV 102 and the CTV 104 are based on anatomic, biological and clinical considerations and do not account for technical factors of treatment. The planning target volume (PTV) 106 is the CTV 104 plus a margin to provide that the prescribed dose is actually absorbed in the CTV 104.

The GTV 102 is based on information from the imaging modalities as well as diagnostic modalities (e.g., pathological reports and clinical examination reports). The CTV 104 encompasses the GTV 102, another region that may have microscopic disease and other areas that may be at a risk from the tumor and may require treatment.

The CTV 104, as represented, includes a margin around the GTV 102. The margin around the GTV 102 may, for example, be 0.5 cm around the GTV 102. In one embodiment, the CTV 104 may be the same as the GTV 102.

The PTV 106, as illustrated in FIG. 1, encompasses the CTV 104. Specifically, the PTV 106 is represented as the CTV 104 and a margin surrounding the CTV 104. The margin surrounding the CTV 104 may be fixed or variable. As an example, the margin around the CTV 104 may be 1 cm to defining the PTV.

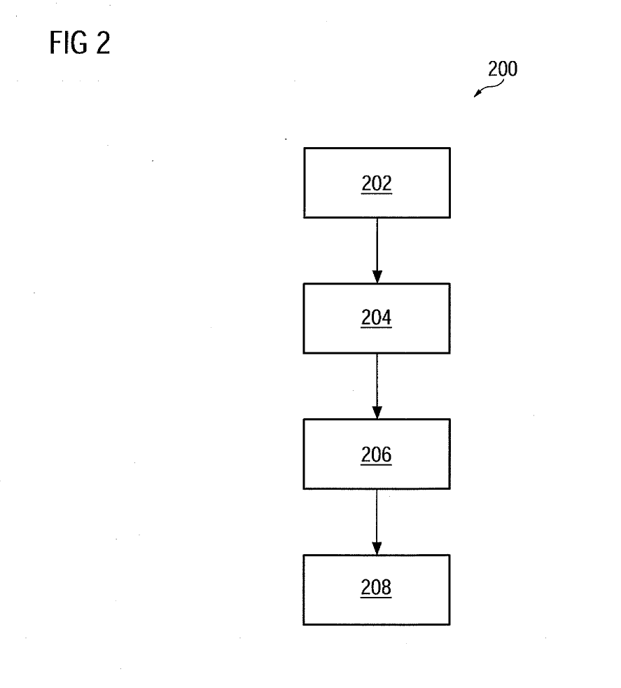

Referring now to FIG. 2, a flowchart depicting one embodiment of a method 200 for visualization of treatment volumes is presented. The treatment volumes include a plurality of volumes (e.g., a first volume and a second volume).

In one embodiment, the first volume may be the GTV and the second volume may be the CTV or the PTV. In another embodiment, the first volume may be the CTV and the second volume may be the PTV. At act 202, a plurality of images of a region of interest is accessed in a subject (e.g., a patient). The plurality of images are parallel to a same plane, and different images of the plurality of images represent different slices of the subject along a normal vector of the plane. The plane may include an axial plane, a coronal plane, or a sagittal plane.

The plurality of images (e.g., medical images) may be acquired by an imaging system such as a CT system or an MR system and may be stored in a repository that may be accessed for treatment planning. Alternatively, the images may also be accessed from a picture archiving and communication system (PACS). In such an embodiment, the PACS may be coupled to a remote system such as, for example, a radiology department information system (RIS), a hospital information system (HIS). Alternatively or additionally, the PACS may be coupled to an internal or external network, so that image data may be accessed from different locations.

The plurality of images include a region of interest that may define boundaries of the malformation (e.g., a tumor) that is to be treated.

At act 204, in the plurality of images, which are parallel to the same plane and represent different slices of the subject along the normal vector of the plane, a cross section of the first treatment volume and a cross section of the second treatment volume in each of the plurality of images is delineated. The delineation act may be performed manually by a user (e.g., a physician, a dosimetrist or a radiologist). Delineation may be done with the help of a marker. Alternatively, delineation of the region of interest may be performed automatically by a treatment planning system that may delineate the cross section of the treatment volumes based upon values entered by the user defining an extent of each of the treatment volumes.

Additionally, the cross section of the first treatment volume and the cross section of the second treatment volume in the image may be delineated by comparing an intensity of a pixel in the image. The delineation of the cross section of the first treatment volume and the cross section of the second treatment volume may also be based upon the specification of a machine used for treatment planning (e.g., dependent on precision tools and machine delivering radiation dose on a region of interest).

In one embodiment, the cross section of the first treatment volume is circumscribed by the cross section of the second treatment volume.

At act 206, the plurality of images are reconstructed to form a three dimensional representation of the treatment volumes from the plurality of images based on the delineated cross sections in the plurality of images. The treatment volumes include one or more volumes (e.g., the first volume and the second volume). In one embodiment, the second volume encompasses the first volume.

Additionally, a third volume and a fourth volume may also be present. The third volume, for example, encompasses the second volume, and the fourth volume encompasses the third volume.

The three dimensional representation may be obtained using an image reconstruction technique from the plurality of two dimensional images. The image reconstruction technique may include techniques such as, for example, a multi planar reconstruction technique or an iterative reconstruction technique.

In one embodiment, the three dimensional representation of the treatment volumes may also be obtained by stacking the plurality of two dimensional images along the same plane and thus obtaining the treatment volumes.

At act 208, a portion of the first treatment volume that is located on a first side of a dividing plane and at least a part of a portion of the second treatment volume that is located on a second side of the dividing plane are displayed in a three dimensional image. The second side opposes the first side. The dividing plane may be a cutting plane dividing the treatment volumes into portions. The dividing plane has two opposite sides. The part of the portion of the second treatment volume is displayed on the second side of the dividing plane, and first treatment volume is located on the first side of the dividing plane.

In one embodiment, the treatment volumes are in the form of a shell. The PTV encompasses the CTV, and the GTV is encompassed by the CTV. The shell view allows visibility of the treatment volumes in sections, while maintaining relative volumetric aspects. The treatment volumes may be rendered on an intensity level of the image acquired. As an example for a CT image, a voxel or volume element in the CT image displays a Hounsfield unit value, thereby providing ease in delineation of volumes.

Alternatively, the treatment volumes may be rendered on dose distribution values that may be computed based on absorbed dose of radiation or an exit dose of radiation. A cut section in the treatment volumes enables the user to detect the correctness of a dose value based on the smoothness of the dose profile, while navigating through planes cutting the treatment volumes.

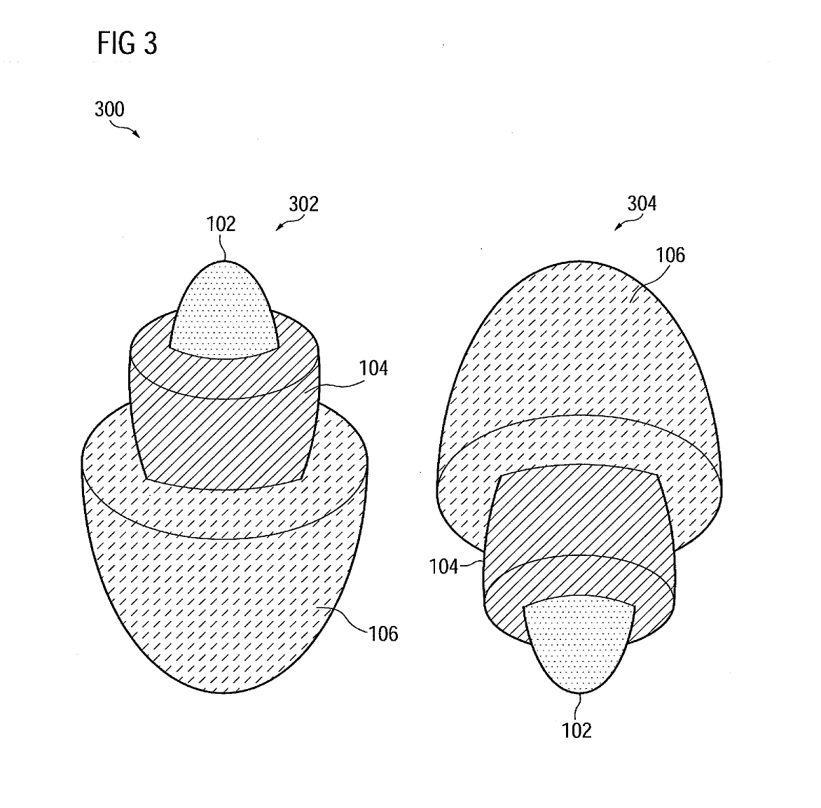

FIG. 3 shows a schematic diagram 300 of three dimensional views of the treatment volumes (e.g., a first treatment volume, a second treatment volume, and a third treatment volume). The PTV encompasses the CTV, which encompasses the GTV. Reference numeral 302 is representative of a first view of the treatment volumes. The treatment volumes are cut by one or more parallel dividing planes (e.g., a first plane, a second plane, and a third plane). The first plane cuts the first treatment volume, such that a portion of the first treatment volume is located on a first side of the dividing plane and at least a part of a portion of the second treatment volume is located on a second side of the dividing plane. The second side is opposite the first side.

Similarly, the second plane and the third plane cut the second treatment volume and the third treatment volume, respectively. The parallel dividing planes may be parametrically represented by f(x, y, z, p1), f(x, y, z, p2), and f(x, y, z, p3), respectively, where p1, p2, p3 are parameters controlling positions of the parallel dividing planes for the first treatment volume, the second treatment volume and the third treatment volume, respectively. The dividing planes reveal frustum of the respective treatment volumes.

In the first view 302, p1≧p2≧p3. This provides that the frustum of the treatment volumes are not obscured by other treatment volumes.

In the second view 304 of the treatment volumes, p1≦p2≦p3. The first view 302 and the second view 304 of the treatment volumes, as depicted in FIG. 3, allow the treatment volumes to be visualized in sections, while maintaining relative volumetric aspects of the volumes. Thus, both 2D and 3D surface information is provided simultaneously.

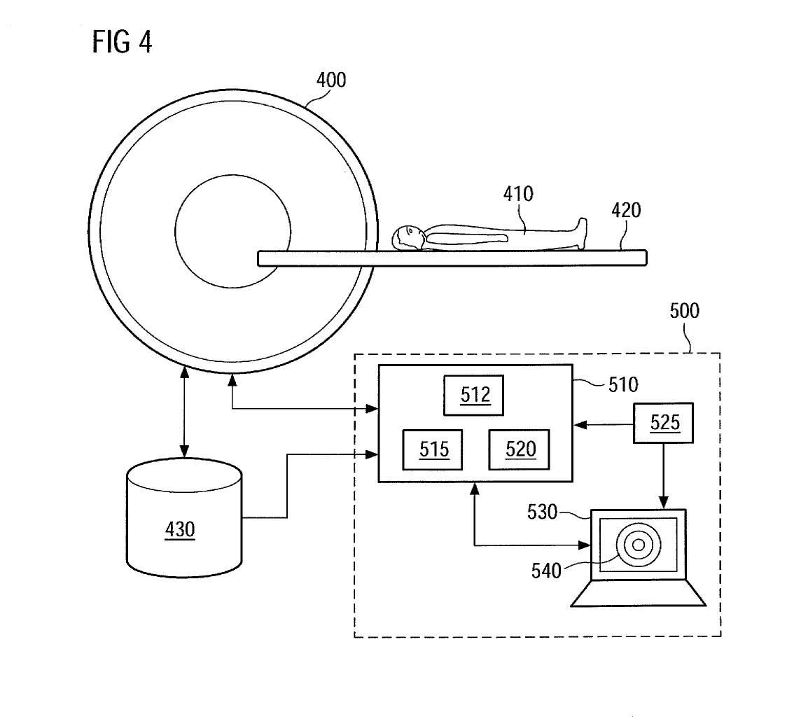

FIG. 4 is a schematic diagram of an exemplary system 500 for visualization of treatment volumes. The system 500 is connected to an image acquisition system 400 or a scanner, such as a CT scanner, an MR scanner, a PET scanner or the like. The image acquisition system 400 includes a bed 420, on which a subject 410 such as a patient lies. The subject 410 is driven into the image acquisition system 400 for acquiring a plurality of medical images of a region of interest of the subject 410. The system 500 may be a standalone computer with software applications running on the standalone computer. Alternatively, the system 500 may be an integral part of the image acquisition system 400.

A data repository 430 is connected to the image acquisition system 400 to store image data. The image data is accessed by a processor 510 of the system 500 for further processing. The processor 510 includes an image accessing module 512 for accessing the medical image data of the subject 410 acquired by the image acquisition system 400. Alternatively or additionally, the image data may be accessed from a picture archiving and communication system (PACS). The PACS may be coupled to a remote system such as a radiology department information system (RIS), a hospital information system (HIS). Alternatively or additionally, the PACS may be coupled to an internal or external network, so that image data may be accessed from different locations. The system 500 includes a display unit 530 to display processed image 540 of the subject 410.

In one embodiment, the image acquisition system 500 acquires images of the subject 410 in the form of slices along a plane (e.g., an axial plane, a coronal plane or a sagittal plane). The image data is stored in the data repository 430. The stored image data is then accessed by the image accessing module 512 in the processor 510 of the exemplary system 500. The processor 510 includes an auto delineation module 515 for automatically delineating a cross section of the first treatment volume and a cross section of the second treatment volume in each image of the plurality of images acquired by the image acquisition system 400. Alternatively, the system 500 also includes an input device 525 coupled to the processor 510 for allowing a user such as a radiologist to manually delineate the cross section of the first treatment volume and the cross section of the second treatment volume in each image of the plurality of images acquired by the image acquisition system 400.

An image reconstruction module 520 in the processor 510 is configured for generating a three dimensional representation of the treatment volumes from the plurality of images by employing three dimensional image reconstruction techniques. The image reconstruction techniques include a multi planar reconstruction technique, an iterative reconstruction technique, or similar reconstruction techniques capable of forming the three dimensional representation from the plurality of two dimensional images. A three dimensional image 540 that includes the three dimensional representation of the treatment volumes is displayed at the display unit 530. The displayed three dimensional image 540 may thereafter be analyzed by the user.

The processor 510 is configured for controlling the transparency of the one or more treatment volumes. The transparency control enables the user to get a better view of the volumes by making the treatment volumes transparent. As an example, by making the first treatment volume transparent, a better view of the second treatment volume is obtained.

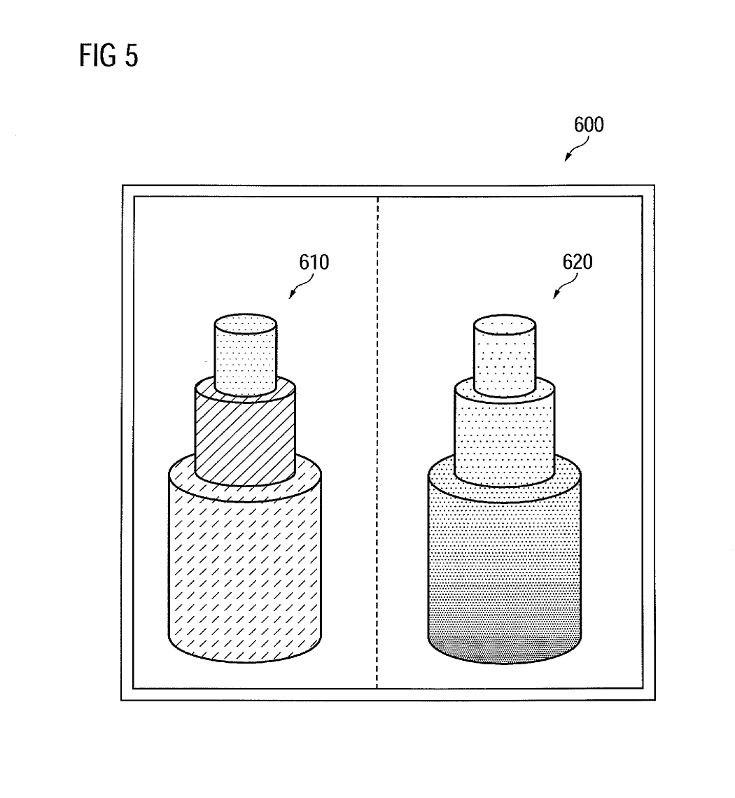

FIG. 5 shows an example displayed image 600. The displayed image 600, which is a three dimensional image, is generated by the display unit 530. The displayed image 600 provides a simultaneous view of treatment volumes, a first view 610 based on an image intensity value of volume elements or voxels and a second view 620 of the treatment volumes depicting a variation in a radiation dose. The radiation dose may be an absorbed dose on the region of interest or an exit dose on the region of interest.

The first view 610 and the second view 620 show the treatment volumes in the form of cylinders. However, the shape of the treatment volumes shows in FIG. 5 is for illustrative purpose only. The treatment volumes may have an irregular shape.

In the second view 620, the density of dots is representative of the dose distribution in the treatment volumes. Alternatively, color variation in the second view 620 may also be used to indicate the dose variation in the treatment volumes.

The exemplary methods and systems for visualization of treatment volumes in medical images, as described hereinabove, have several advantages. The present embodiments provide an intuitive view of the treatment volumes and aid an oncologist to analyze and plan a better treatment. The present embodiments also provide an efficient way of studying an exit dosimetry, whereby the dose distribution in the treatment volumes is determined.

While the present invention has been described above by reference to various embodiments, it should be understood that many changes and modifications can be made to the described embodiments. It is therefore intended that the foregoing description be regarded as illustrative rather than limiting, and that it be understood that all equivalents and/or combinations of embodiments are intended to be included in this description.

1. A method for visualization of at least a first treatment volume and a second treatment volume, the second treatment volume encompassing the first treatment volume, the method comprising:
accessing a plurality of images of a region of interest in a subject, wherein the plurality of images represent different slices that are parallel to a plane, and wherein the different slices of the subject are along a normal vector of the plane;
delineating a cross section of the first treatment volume and a cross section of the second treatment volume in each image of the plurality of images;
reconstructing a three dimensional representation of the first treatment volume and the second treatment volume from the plurality of images based on the delineated cross sections in the plurality of images; and
displaying, in a three dimensional image, a portion of the first treatment volume that is located on a first side of a dividing plane and displaying, in the three dimensional image, at least a part of a portion of the second treatment volume that is located on a second side of the dividing plane, wherein the second side is opposite the first side.

2. The method according to claim 1, wherein the plurality of images are acquired in an axial plane, a sagittal plane or a coronal plane.

3. The method according to claim 1, wherein the first treatment volume is a gross tumor volume and the second treatment volume is a clinical target volume or a planning target volume.

4. The method according to claim 1, wherein the first treatment volume is a clinical target volume and the second treatment volume is a planning target volume.

5. The method according to claim 1, wherein the plurality of images of the subject computed tomography images, magnetic resonance images, positron emission tomography images, or a combination thereof.

6. The method according to claim 1, wherein the first treatment volume and the second treatment volume are reconstructed using a three dimensional image reconstruction technique.

7. The method according to claim 6, wherein the three dimensional image reconstruction technique comprises a multi planar reconstruction technique or an iterative reconstruction technique.

8. The method according to claim 1, wherein the delineation of the cross sections is performed manually by a user.

9. The method according to claim 1, wherein the delineation of the cross sections is performed automatically by a computer program.

10. The method according to claim 1, wherein the cross sections are delineated based upon an intensity value of a voxel in the three dimensional image.

11. A system for visualization of at least a first treatment volume and a second treatment volume, the second treatment volume encompassing the first treatment volume, the system comprising:
a processor coupled to an image acquisition system operable to acquire a plurality of images of a region of interest in a subject, the processor configured for accessing the plurality of images of the region of interest in the subject, wherein the plurality of images represent different slices that are parallel to a plane, and wherein the different slices of the subject are along a normal vector of the plane;
delineating a cross section of the first treatment volume and a cross section of the second treatment volume in each image of the plurality of images; and
reconstructing a three dimensional representation of the first treatment volume and the second treatment volume from the plurality of images based on the delineated cross sections in the plurality of images; and
a display unit operable to display, in a three dimensional image, a portion of the first treatment volume that is located on a first side of a dividing plane and displaying, in the three dimensional image, at least a part of a portion of the second treatment volume that is located on a second side of the dividing plane, wherein the second side is opposite the first side.

12. The system according to claim 11, wherein the first treatment volume is a gross tumor volume, and the second treatment volume is a clinical target volume or a planning target volume, or
wherein the first treatment volume is the clinical target volume, and the treatment second volume is the planning target volume.

13. The system according to claim 11, wherein the processor comprises an image accessing module, the image accessing module being configured for accessing the plurality of images of the region of interest in the subject.

14. The system according to claim 11, further comprising an input device operable to delineate the cross section of the first treatment volume and the cross section of the second treatment volume in each image of the plurality of images.

15. The system according to claim 11, wherein the processor comprises an auto delineation module, the auto delineation module being configured for automatically delineating the cross section of the first treatment volume and the cross section of the second treatment volume in each image of the plurality of images.

16. The system according to claim 11, wherein the display unit is configured to simultaneously display the first treatment volume and the second treatment volume based on a variation in intensity in the three dimensional image.

17. The system according to claim 11, wherein the display unit is further configured to display the first treatment volume and the second treatment volume depicting a variation in a radiation dose.

18. The system according to claim 11, wherein the processor comprises an image reconstruction module, the image reconstruction module being configured for generating the three dimensional representation of the first treatment volume and the second treatment volume from the plurality of images based on the delineated cross sections in the plurality of images.

19. In a non-transitory computer readable storage medium having stored therein data representing instructions executable by a processor for visualizing at least a first treatment volume and a second treatment volume, the second treatment volume encompassing the first treatment volume, the instructions comprising:
accessing a plurality of images of a region of interest in a subject, wherein the plurality of images represent different slices that are parallel to a plane, and wherein the different slices of the subject are along a normal vector of the plane;
delineating a cross section of the first treatment volume and a cross section of the second treatment volume in each image of the plurality of images;
reconstructing a three dimensional representation of the first treatment volume and the second treatment volume from the plurality of images based on the delineated cross sections in the plurality of images; and
displaying, in a three dimensional image, a portion of the first treatment volume that is located on a first side of a dividing plane and displaying, in the three dimensional image, at least a part of a portion of the second treatment volume that is located on a second side of the dividing plane, wherein the second side is opposite the first side.
